(19) United States
(12) Patent Application Publication
(10) Pub. No.: US 2018/0000364 A1
(43) Pub. Date: Jan. 4, 2018

(54) LIGHT-EMITTING ELEMENT, DETECTION DEVICE, AND PROCESSING APPARATUS

(71) Applicant: Kabushiki Kaisha Toshiba, Minato-ku (JP)

(72) Inventors: Toshiya YONEHARA, Kawasaki (JP); Tomio ONO, Yokohama (JP); Tomoaki SAWABE, Taito (JP)

(73) Assignee: Kabushiki Kaisha Toshiba, Minato-ku (JP)

(21) Appl. No.: 15/705,921

(22) Filed: Sep. 15, 2017

Related U.S. Application Data
(63) Continuation of application No. PCT/JP2015/061694, filed on Apr. 16, 2015.

(51) Int. Cl. A61B 5/024; A61B 5/00; H01L 27/32; G01F 1/66; H01L 51/50; H01L 51/00; H01L 51/52; H05B 37/02

(52) U.S. Cl. CPC A61B 5/02427; H01L 51/524; H01L 51/5268; A61B 5/6803; H05B 37/0227; G01F 1/661; H01L 51/5206; H01L 51/5012; H01L 51/0096; H01L 27/3227; H01L 51/5221; A61B 5/6824; A61B 5/6826; A61B 5/6828; A61B 5/6831; H01L 2251/558

(57) ABSTRACT

According to one embodiment, a light-emitting element includes a substrate, a first electrode, a second electrode, and a light-emitting layer. The substrate is light-transmissive. The second electrode is provided between the first electrode and a portion of the substrate. The second electrode is light-transmissive. A light-emitting layer is provided between the first electrode and the second electrode. The substrate includes a first region and a second region. The first region overlaps at least a portion of the light-emitting layer in a first direction, the first direction is from the second electrode toward the first electrode. The second region is provided around the first region along a plane perpendicular to the first direction. The substrate has an opening provided in at least a portion of the second region.

### CROSS-REFERENCE TO RELATED APPLICATIONS

This is a continuation application of International Application PCT/JP2015/061694, filed on Apr. 16, 2015; the entire contents of which are incorporated herein by reference.

### FIELD

Embodiments described herein relate generally to a light-emitting element, a detection device, and a processing apparatus.

### BACKGROUND

There is a detection device that utilizes a light-emitting element. For example, there is a detection device that detects a biological signal by irradiating light radiated from a light-emitting element onto a living body. It is desirable to develop a light-emitting element suited to the detection of a pulse wave having a faint output signal.

### BRIEF DESCRIPTION OF THE DRAWINGS

FIG. 1 is a schematic bottom view illustrating an example of a light-emitting element according to a first embodiment;

FIG. 2 is a schematic cross-sectional view illustrating an A-A′ cross section of FIG. 1;

FIG. 3A and FIG. 3B are schematic views illustrating examples of optical paths of light-emitting elements;

FIG. 4 is a schematic cross-sectional view illustrating another example of the light-emitting element according to the first embodiment;

FIG. 5A to FIG. 5C and FIG. 6A to FIG. 6D are schematic cross-sectional views illustrating portions of the light-emitting element according to the embodiment;

FIG. 7A, FIG. 7B, FIG. 8A, and FIG. 8B are schematic cross-sectional views illustrating other examples of the light-emitting element according to the first embodiment;

FIG. 9A and FIG. 9B illustrate simulation results of the light-emitting element according to the first embodiment;

FIG. 10A and FIG. 10B illustrate other simulation results of the light-emitting element according to the first embodiment;

FIG. 11A, FIG. 11B, FIG. 12A, FIG. 12B, FIG. 13A, FIG. 13B, FIG. 14A, and FIG. 14B are schematic bottom views illustrating other examples of the light-emitting element according to the first embodiment;

FIG. 15 is a schematic plan view illustrating an example of a light-emitting element according to a second embodiment;

FIG. 16 is a schematic cross-sectional view illustrating an A-A′ cross section of FIG. 15;

FIG. 17A and FIG. 17B are schematic views illustrating an example of a light-emitting element according to a third embodiment;

FIG. 18A and FIG. 18B are schematic cross-sectional views illustrating other examples of the light-emitting element according to the third embodiment;

FIG. 19A and FIG. 19B are schematic views illustrating an example of a light-emitting element according to a fourth embodiment;

FIG. 20A and FIG. 20B are schematic cross-sectional views illustrating an example of a detection device according to a fifth embodiment;

FIG. 21 and FIG. 22 are schematic views illustrating an example of a processing apparatus including the light-emitting element according to the embodiment;

FIG. 23A to FIG. 26B are schematic views illustrating a pulse wave being measured using the light-emitting element according to the embodiment;

FIG. 27A to FIG. 27C are schematic views illustrating processing apparatuses including the light-emitting element according to the embodiment;

FIG. 28A to FIG. 28E are schematic views illustrating applications of processing apparatuses including the light-emitting element according to the embodiment; and

FIG. 29 is a schematic view illustrating a system including the processing apparatuses illustrated in FIGS. 28A to 28E.

### DETAILED DESCRIPTION

According to one embodiment, a light-emitting element includes a substrate, a first electrode, a second electrode, and a light-emitting layer. The substrate is light-transmissive. The second electrode is provided between the first electrode and a portion of the substrate. The second electrode is light-transmissive. A light-emitting layer is provided between the first electrode and the second electrode. The substrate includes a first region and a second region. The first region overlaps at least a portion of the light-emitting layer in a first direction, the first direction is from the second electrode toward the first electrode. The second region is provided around the first region along a plane perpendicular to the first direction. The substrate has an opening provided in at least a portion of the second region.

Embodiments of the invention will now be described with reference to the drawings.

The drawings are schematic or conceptual; and the relationships between the thicknesses and widths of portions, the proportions of sizes between portions, etc., are not necessarily the same as the actual values thereof. The dimensions and/or the proportions may be illustrated differently between the drawings, even in the case where the same portion is illustrated.

In the drawings and the specification of the application, components similar to those described thereinabove are marked with like reference numerals, and a detailed description is omitted as appropriate.

### First Embodiment

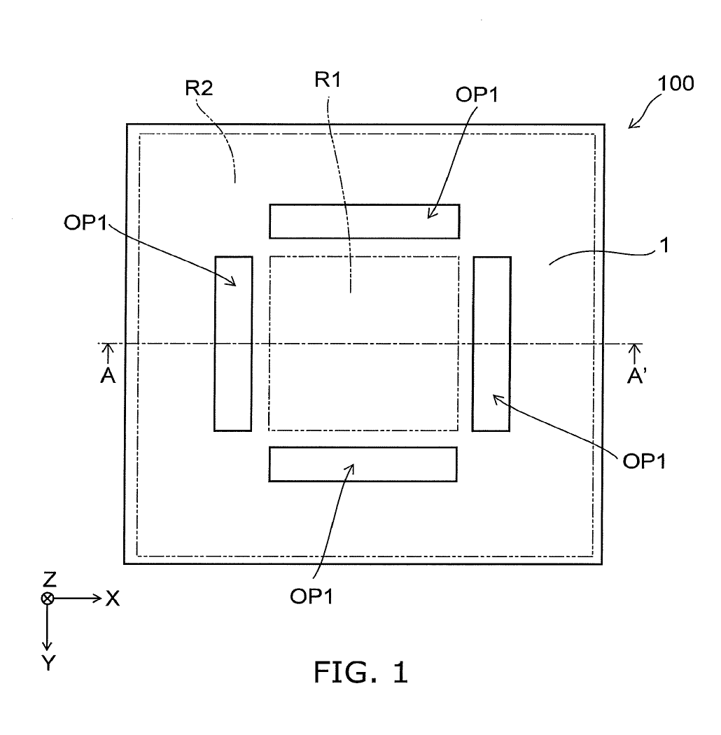

FIG. 1 is a schematic bottom view illustrating an example of a light-emitting element according to a first embodiment.

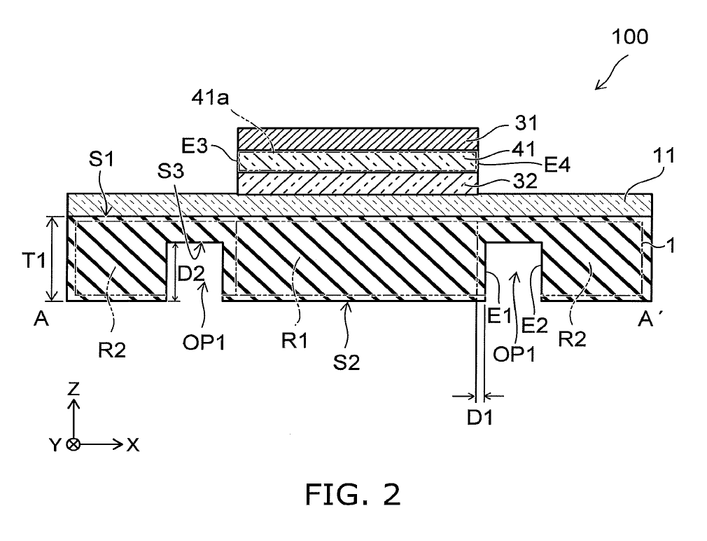

FIG. 2 is a schematic cross-sectional view illustrating an A-A′ cross section of FIG. 1.

As illustrated in FIG. 1 and FIG. 2, the light-emitting element 100 includes a substrate 1, a first layer 11, a second electrode 32, a light-emitting layer 41, and a first electrode 31. For example, the light-emitting element 100 is used to detect a biological signal such as a pulse wave, etc.

A direction from the second electrode 32 toward the first electrode 31 is taken as a first direction. The first direction is, for example, a Z-direction. Two mutually-perpendicular directions perpendicular to the first direction are taken as a second direction and a third direction. For example, the second direction is an X-direction; and the third direction is a Y-direction.

As illustrated in FIG. 1, the substrate 1 includes a first region R1 and a second region R2. The first region R1 overlaps a light-emitting region 41a in the first direction. The light-emitting region 41a is a region of at least a portion of the light-emitting layer 41. The light-emitting region 41a is positioned between the first electrode 31 and the second electrode 32 in the first direction. The second region R2 is provided around the first region R1 along a plane perpendicular to the first direction.

The substrate 1 has an opening OP1. The opening OP1 is provided in the second region R2. In the example illustrated in FIG. 1 and FIG. 2, multiple openings OP1 are provided in the substrate 1. At least a portion of the multiple openings OP1 may be provided in the first region R1. At least one of the multiple openings OP1 is provided along the boundary between the first region R1 and the second region R2.

At least one of the multiple openings OP1 is, for example, a trench. In the example illustrated in FIG. 1 and FIG. 2, the substrate 1 has at least one trench extending along the second direction and at least one trench extending along the third direction.

For example, one of the openings includes a first end E1 and a second end E2. The light-emitting layer 41 includes a third end E3 and a fourth end E4. The direction from the first end E1 toward the second end E2 is aligned with the second direction. The direction from the third end E3 toward the fourth end E4 is aligned with the second direction. The position in the second direction of the first end E1 is between the position in the second direction of the third end E3 and the position in the second direction of the second end E2. The position in the second direction of the fourth end E4 is between the position in the second direction of the third end E3 and the position in the second direction of the second end E2. Preferably, the direction from the first end E1 toward the fourth end E4 is aligned with the first direction.

As illustrated in FIG. 2, the substrate 1 has a first surface S1, a second surface S2, and a third surface S3. The first to third surfaces S1 to S3 are aligned with a plane perpendicular to the first direction. The first surface S1 is a surface on the first electrode 31 side of the substrate 1. The second surface S2 is a surface on the side opposite to the first surface S1. The third surface S3 is a surface in the opening OP1. The position in the first direction of the third surface S3 is between the position in the first direction of the second surface S2 and the position in the first direction of the first surface S1.

The opening OP1 is provided in the second surface S2. For example, a portion of the substrate 1 is positioned between the opening OP1 and the first surface S1.

At least a portion of the first layer 11 is provided between the first electrode 31 and at least a portion of the substrate 1 in the first direction. The first layer 11 is configured to modify the travel direction inside the layer of the first layer 11 of the light incident on the first layer 11. The first layer 11 is provided as necessary and may be omitted. In the embodiment, it is favorable for the first layer 11 to be provided. Thereby, for example, light can be radiated efficiently to the outside from the substrate 1.

The second electrode 32 is provided between the first electrode 31 and at least a portion of the substrate 1 in the first direction. The light-emitting layer 41 is provided between the first electrode 31 and the second electrode 32 in the first direction.

Light is radiated from the light-emitting layer 41 by carriers being injected into the light-emitting layer 41 from the first electrode 31 and the second electrode 32. The light-emitting layer 41 includes, for example, an organic substance. The noise is smaller for the light radiated from a light-emitting element using a light-emitting layer including an organic substance than for the light radiated from a light-emitting element using a light-emitting layer including an inorganic compound. Therefore, the light that is radiated from the light-emitting element using the light-emitting layer including the organic substance is suited to applications that detect a detection object such as a pulse wave, etc., in which the signal that is output is faint.

For example, the substrate 1, the first layer 11, and the second electrode 32 transmit the light radiated from the light-emitting layer 41. The substrate 1, the first layer 11, and the second electrode 32 are light-transmissive. The first electrode 31 is light-reflective. The first electrode 31 reflects the light radiated from the light-emitting layer 41. The reflectance of the first electrode 31 is higher than the reflectance of the substrate 1, higher than the reflectance of the first layer 11, and higher than the reflectance of the second electrode 32.

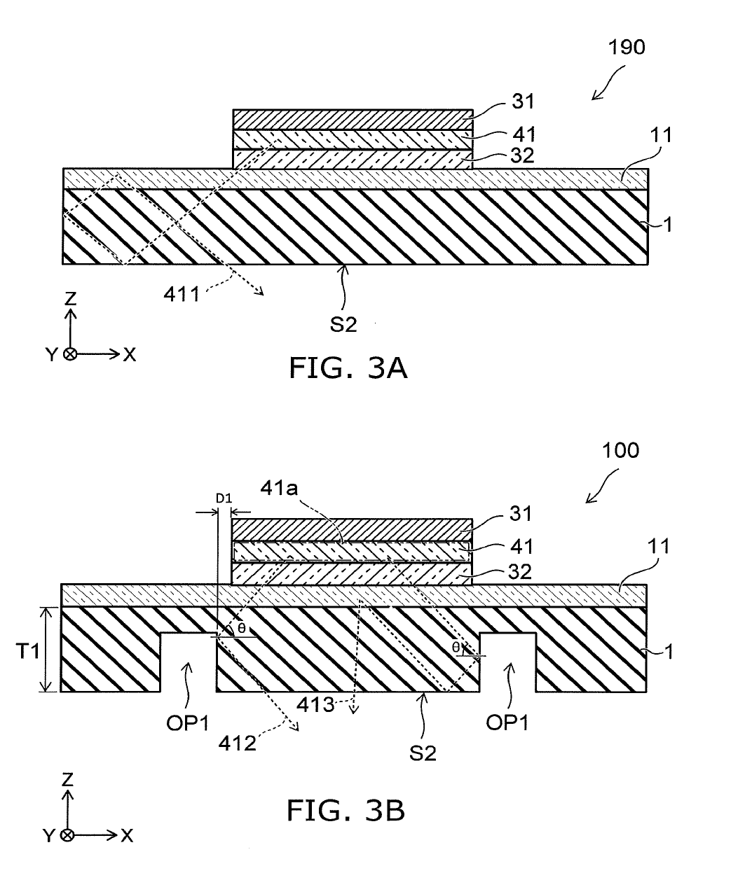

FIG. 3A and FIG. 3B are schematic views illustrating examples of optical paths of light-emitting elements.

FIG. 3A illustrates an example of the optical path of a light-emitting element 190 according to a reference example. FIG. 3B illustrates an example of the optical path of the light-emitting element 100 according to the first embodiment.

In the light-emitting element 190, for example, light 411 that is radiated from the light-emitting layer 41 passes through the first layer 11 and is incident on the substrate 1. The light 411 is reflected by the second surface S2 of the substrate 1 and travels in the in-plane direction through the interior of the substrate 1. After being reflected by the side surface of the substrate 1, the light 411 is scattered by the first layer 11. The travel direction of the light 411 is modified by being scattered by the first layer 11; and the light 411 is emitted to the outside from the second surface S2 of the substrate 1.

On the other hand, in the light-emitting element 100, for example, light 412 that is radiated from the light-emitting layer 41 passes through the first layer 11 and is incident on the substrate 1. The light 412 is reflected by the interface between the substrate 1 and the opening OP1 and is emitted to the outside from the second surface S2 of the substrate 1.

Or, in the light-emitting element 100, light 413 that is radiated from the light-emitting layer 41 is incident on the substrate 1, is reflected by the interface between the substrate 1 and the opening OP1, is reflected by the second surface 52, and subsequently is incident on the first layer 11. The travel direction of the light 413 is modified by being scattered by the first layer 11; and the light 413 is emitted to the outside from the second surface S2 of the substrate 1.

As illustrated in FIG. 3B, by providing the opening OP1 in at least a portion of the second region R2, the distance that the light radiated from the light-emitting layer 41 travels through the interior of the substrate 1 can be shortened. By shortening the distance that the light travels in the interior of the substrate 1, the absorption of the light in the substrate 1 is reduced. Therefore, it is possible to increase the amount of the light radiated to the outside from the substrate 1. By employing such a configuration, the amount of the light radiated in the space overlapping the light-emitting region 41a in the first direction can be increased. According to the embodiment, a light-emitting element is provided that is suited to applications that detect a biological signal such as a pulse wave, etc., in which it is desirable to irradiate light in a designated region.

In the example illustrated in FIG. 1 and FIG. 2, the opening OP1 is provided in the second surface S2 of the substrate 1. By employing such a configuration, it is possible to easily form the contact structures of the first electrode 31 and the second electrode 32 on the first surface S1 of the substrate 1 while increasing the amount of the light radiated in the space overlapping the light-emitting region 41a in the first direction.

In the embodiment, for example, the light that reaches the side surface of the substrate 1 is reflected by the side surface similarly to the light 412; and the travel direction of the light is modified toward the interior of the substrate 1. Thereby, for example, the light is radiated efficiently to the outside from the second surface 52 of the substrate 1. To this end, it is desirable for an incident angle θ of the light on the side surface, a thickness T1, and a distance D1 to satisfy the following Formula (1).

T1≧D1×tanθ (1)

The thickness T1 is the thickness in the first direction of the substrate 1. The distance D1 is the distance in the second direction between the light-emitting region 41a and the opening OP1. For example, the distance D1 is equal to the distance in the second direction between the first region R1 and the opening OP1.

The critical angle at which total internal reflection occurs is determined by the refractive index n of the substrate 1. The light that is incident on the side surface of the substrate 1 at an angle that is smaller than the critical angle undesirably is emitted to the outside from the side surface of the substrate 1. Therefore, the minimum angle of θ in Formula (1) is the critical angle. Using the critical angle θ_{c}, Formula (1) is represented as in the following Formula (2).

T1≧D1×tanθ_{c} (2)

For example, it is desirable for the thickness T1 and the distance D1 to satisfy the following relationship. Thereby, the proportion of the light radiated from the light-emitting region 41a that is emitted to the outside can be improved.

$$D 1 ≦ \frac{T 1}{tan {θ}_{c}} , {θ}_{c} = arcsin \frac{1}{n} \qquad ( 3 )$$

It is more desirable for the light reflected by the side surface of the substrate 1 to be incident on the first layer 11 and for the travel direction of the light to be modified similarly to the light 413. Therefore, it is more desirable for the distance D1, the thickness T1, and the incident angle e on the side surface of the light radiated from the end portion of the light-emitting region 41a to satisfy the following Formula (4).

T1≧2×D1×tanθ (4)

The light that is incident on the side surface of the substrate 1 at an angle that is smaller than the critical angle undesirably is emitted outside the substrate 1. Therefore, the minimum angle of θ in Formula (4) is the critical angle. By using the critical angle θ_{c}, Formula (4) is represented as in the following Formula (5).

T1≧2×D1×tanθ_{c} (5)

For example, it is more desirable for the thickness T1 and the distance D1 to satisfy the following relationship. Thereby, the proportion of the light radiated from the light-emitting region 41a that is emitted to the outside can be improved.

$$T 1 ≦ \frac{D 1}{2 tan {θ}_{c}} , {θ}_{c} = arcsin \frac{1}{n} \qquad ( 6 )$$

Examples of the components will now be described.

The substrate 1 includes, for example, glass. The refractive index of the substrate 1 is, for example, not less than 1.4 and not more than 2.2. The thickness T1 along the first direction of the substrate 1 is, for example, 0.05 to 2.0 mm.

The first electrode 31 includes, for example, at least one of aluminum, silver, or gold. The first electrode 31 includes, for example, an alloy of magnesium and silver.

The second electrode 32 includes, for example, ITO (Indium Tin Oxide). The second electrode 32 may include, for example, a conductive polymer such as PEDOT: PSS, etc. The second electrode 32 may include, for example, a metal (including, for example, at least one of aluminum or silver). In the case where the second electrode 32 includes a metal, it is favorable for the thickness of the second electrode 32 to be 5 to 20 nm.

The light-emitting layer 41 includes, for example, at least one of Alq₃ (tris(8-hydroxyquinolinolato)aluminum), F8BT (poly(9,9-dioctylfluorene-co-benzothiadiazole)), or PPV (polyparaphenylene vinylene).

Or, the light-emitting layer 41 may include a host material and a dopant material. The host material includes, for example, at least one of CBP (4,4′-N,N′-bis dicarbazolyl-biphenyl), BCP (2,9-dimethyl-4,7 diphenyl-1,10-phenanthroline), TPD (2,9-dimethyl-4,7 diphenyl-1,10-phenanthroline), PVK (polyvinyl carbazole), or PPT (poly(3-phenylthiophene)). The dopant material includes, for example, at least one of Flrpic (iridium(III)-bis(4,6-di-fluorophenyl)-pyridinate-N,C2′-picolinate), Ir(ppy)₃ (tris(2-phenylpyridine)iridium), or FIr6 (bis(2,4-d ifluorophenylpyridinato)-tetrakis(1-pyrazolyl)borate-iridium(III)).

The light that is radiated from the light-emitting layer 41 is, for example, visible light. For example, the light that is radiated from the light-emitting layer 41 is one of red, orange, yellow, green, or blue light or a combination of such light. The light that is radiated from the light-emitting layer 41 may be ultraviolet light or infrared light.

In a plane perpendicular to the first direction, the configuration of the first electrode 31, the configuration of the light-emitting layer 41, and the configuration of the second electrode 32 are, for example, polygons (of which the corners may be curves) or circles (including flattened circles). These configurations are arbitrary.

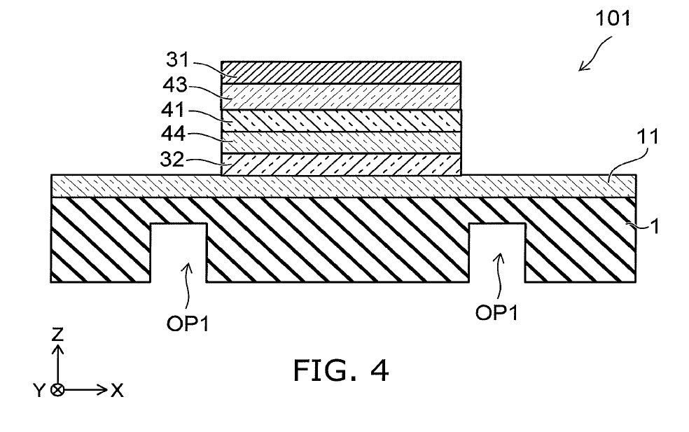

FIG. 4 is a schematic cross-sectional view illustrating another example of the light-emitting element according to the first embodiment. As in the light-emitting element 101 illustrated in FIG. 4, a third layer 43 may be provided between the first electrode 31 and the light-emitting layer 41; and a fourth layer 44 may be provided between the second electrode 32 and the light-emitting layer 41.

The third layer 43 functions as, for example, an electron injection layer. The third layer 43 may function as an electron transport layer. Or, the third layer 43 may include a layer that functions as an electron injection layer and a layer that functions as an electron transport layer.

The third layer 43 includes, for example, at least one of Alq₃, BAlq, POPy₂, Bphen, or 3TPYMB. In such a case, for example, the third layer 43 functions as an electron transport layer.

The third layer 43 includes, for example, at least one of LiF, CsF, Ba, or Ca. In such a case, the third layer 43 functions as, for example, an electron injection layer.

The fourth layer 44 functions as, for example, a hole injection layer. The fourth layer 44 may function as a hole transport layer. Or, the fourth layer 44 may include a layer that functions as a hole injection layer and a layer that functions as a hole transport layer.

The fourth layer 44 includes, for example, α-NPD, TAPC, m-MTDATA, TPD, or TCTA. In such a case, for example, the fourth layer 44 functions as a hole transport layer.

The material of the fourth layer 44 includes, for example, at least one of PEDPOT: PPS, CuPc, or MoO₃. In such a case, for example, the fourth layer 44 functions as a hole injection layer.

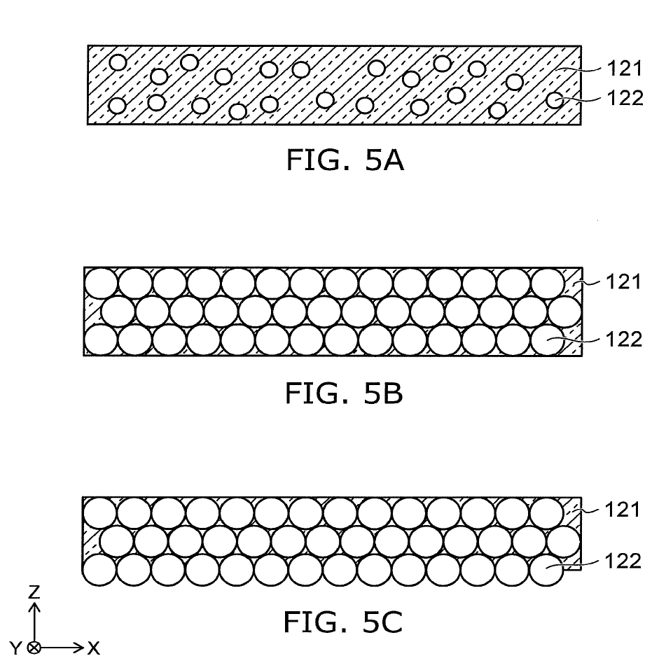

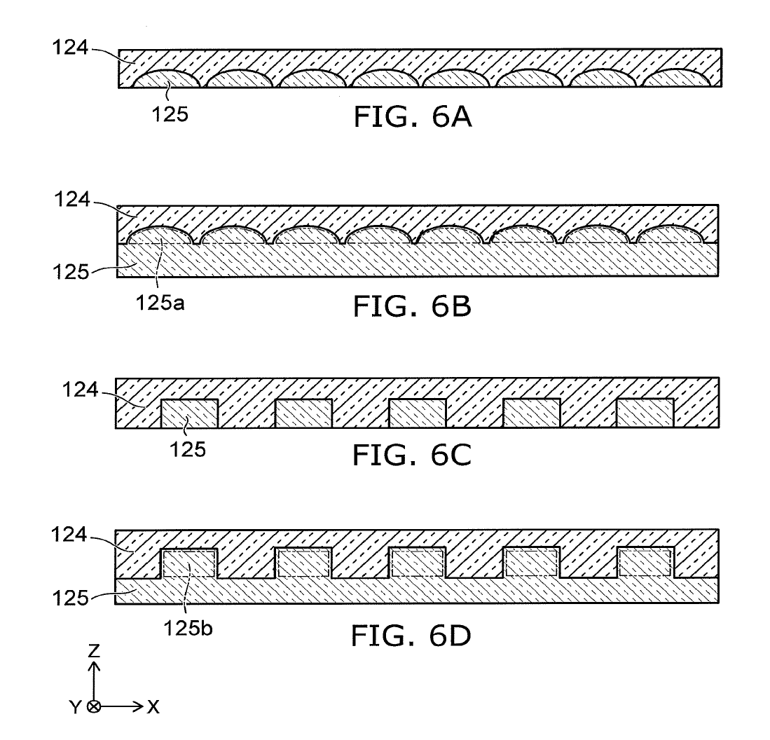

FIG. 5A to FIG. 5C and FIG. 6A to FIG. 6D are schematic cross-sectional views illustrating portions of the light-emitting element according to the embodiment.

FIG. 5A to FIG. 5C illustrate the first layer 11. In the examples illustrated in these drawings, at least a portion of the light incident on the first layer 11 is scattered in the first layer 11. In the examples illustrated in FIG. 6A to FIG. 6D, at least a portion of the light incident on the first layer 11 is refracted in the first layer 11.

As illustrated in FIG. 5A to FIG. 5C, the first layer 11 includes, for example, a support portion 121 and multiple particles 122. For example, the support portion 121 spreads along the first surface perpendicular to the first direction.

In the example illustrated in FIG. 5A, the multiple particles 122 are provided to be separated from each other. The support portion 121 is provided around each of the multiple particles 122. In the example illustrated in FIG. 5B, at least a portion of the multiple particles 122 is provided to be in contact with each other; and the support portion 121 is provided around each of the multiple particles 122.

In the example illustrated in FIG. 5C, a portion of the multiple particles 122 is exposed outside the support portion 121. The support portion 121 is provided around at least a portion of each of the multiple particles 122. A portion of the support portion 121 is provided around a portion of the particles 122 exposed outside the support portion 121. Another portion of the support portion 121 is provided around another portion of the multiple particles 122.

The support portion 121 includes, for example, at least one of a resin or a polymer. The polymer includes, for example, polysiloxane, polyimide, polymethyl methacrylate, etc. At least one of the multiple particles 122 includes, for example, at least one of silica, polystyrene, zirconium oxide, or titanium oxide. Voids may be provided instead of the particles 122. It is desirable for the absolute value of the difference between the refractive index of the support portion 121 and the refractive index of at least one of the multiple particles 122 to be 0.1 or more. More desirably, the absolute value of the difference of these refractive indexes is 0.2 or more. By setting the absolute value of the difference of these refractive indexes to be 0.1 or more, for example, a high scattering property for the light incident on the first layer 11 is obtained. When the difference of the refractive indexes is large, the scattering property due to the particles 122 is high. When the difference of the refractive indexes is large, a high scattering property is obtained easily even in the case where the density of the particles 122 is low.

The particle size of the particle 122 may be, for example, a maximum of 100 μm. In the case where the first layer 11 is made by spin coating, the thickness of the support portion 121 is a maximum of about 10 μm due to of the constraints of the viscosity of the material. Accordingly, in the case of such a support portion 121, it is favorable for the particle size of the particle 122 to be a maximum of 10 μm. It is desirable for the particle size of at least one particle 122 of the multiple particles 122 to be greater than 1/10 of the peak wavelength of the light. In the case where the particle size is greater than 1/10 of the peak wavelength of the light, the scattering follows a Mie scattering model.

In the case where the particle size of the particle 122 is sufficiently smaller than the wavelength of the light, the spatial resolution between the support portion 121 and the particles 122 disappears from the perspective of the light. In other words, in such a case, from the perspective of the light, the first layer 11 is a layer having the average refractive index of the refractive index of the support portion 121 and the refractive index of the particle 122; and the scattering ability of the first layer 11 for the light decreases.

As illustrated in FIG. 6A to FIG. 6D, the first layer 11 includes, for example, a first portion 124 and a second portion 125. The second portion 125 is provided between the first portion 124 and the substrate 1. The refractive index of the second portion 125 is lower than the refractive index of the first portion 124.

In the example illustrated in FIG. 6A, the second portion 125 is multiply provided in the second direction. The second portions 125 also may be multiply provided in the third direction. Or, the second portions 125 may extend in the third direction.

The first portion 124 spreads along a plane perpendicular to the first direction. Each of the second portions 125 is surrounded with the first portion 124 along the plane perpendicular to the first direction. The second portions 125 have hemispherical configurations. Therefore, the thickness in the first direction of the first portion 124 changes periodically and continuously in the second direction.

Or, as illustrated in FIG. 6B, the second portion 125 may spread along a plane perpendicular to the first direction. The second portion 125 includes a hemispherical portion 125a that is surrounded with the first portion 124. For example, the hemispherical portion 125a is multiply provided in the second direction and the third direction.

As illustrated in FIG. 6C, the second portion 125 may have a surface along the first direction and a surface along the second direction. The thickness along the first direction of the first portion 124 changes periodically in a staircase configuration.

Or, the second portion 125 may spread along the first surface as illustrated in FIG. 6D. The second portion 125 includes a protruding portion 125b having a surface along the first direction and a surface along the second direction. For example, the protruding portion 125b is multiply provided in the second direction; and each of the protruding portions 125b extends in the third direction.

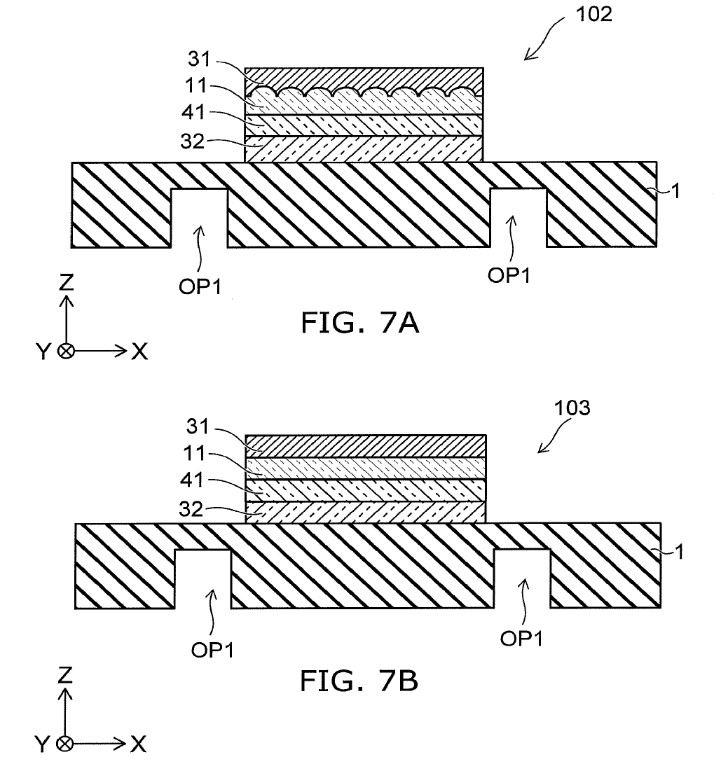

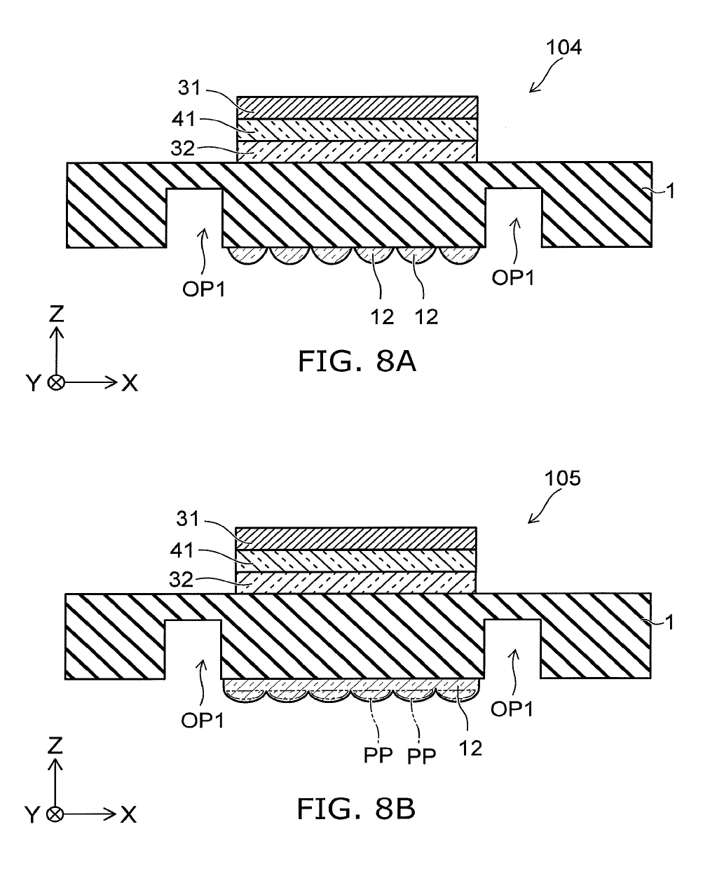

FIG. 7A and FIG. 7B are schematic cross-sectional views illustrating other examples of the light-emitting element according to the first embodiment. The first layer 11 may be provided somewhere other than between the substrate 1 and the second electrode 32 such as in a light-emitting element 102 illustrated in FIG. 7A or a light-emitting element 103 illustrated in FIG. 7B. In the examples illustrated in these drawings, the first layer 11 is provided between the first electrode 31 and the light-emitting layer 41.

The first layer 11 may be provided both between the substrate 1 and the second electrode 32 and between the first electrode 31 and the light-emitting layer 41. The first layer 11 is provided in at least one of a first position in the first direction between the substrate 1 and the second electrode 32 or a second position in the first direction between the first electrode 31 and the light-emitting layer 41.

In the example illustrated in FIG. 7A, the interface between the first layer 11 and the first electrode 31 has an uneven structure. As one specific example, the distance between the second electrode 32 and the interface between the first layer 11 and the first electrode 31 changes periodically in the second direction. In the example, the first layer 11 may function as an electron injection layer or an electron transport layer. Or, the first layer 11 may include a layer that functions as an electron injection layer and a layer that functions as an electron transport layer.

In the example illustrated in FIG. 7B, the first layer 11 has the structure illustrated in any of FIG. 5A to FIG. 5C. In such a case, a conductive material is included in the support portion 121 included in the first layer 11. The support portion 121 that is included in the first layer 11 functions as, for example, an electron transport layer. Or, the support portion 121 that is included in the first layer 11 functions as, for example, an electron injection layer.

FIG. 8A and FIG. 8B are schematic cross-sectional views illustrating other examples of the light-emitting element according to the first embodiment. A second layer 12 may be provided instead of the first layer 11 as in a light-emitting element 104 illustrated in FIG. 8A or a light-emitting element 105 illustrated in FIG. 8B. By providing the second layer 12, the amount of the light radiated outside the substrate 1 can be increased compared to the case where there is no second layer 12.

The light-emitting element may include both the first layer 11 and the second layer 12.

In the example illustrated in FIG. 8A, multiple second layers 12 are provided on the lower surface of the substrate 1. The second layers 12 protrude from the lower surface of the substrate 1 toward a fourth direction, where the fourth direction is from the first electrode 31 toward the second electrode 32. The fourth direction is, for example, the reverse direction of the Z-direction illustrated in FIGS. 8A and 8B.

In an example, the upper surface of the second layer 12 is aligned with a plane perpendicular to the fourth direction; and the lower surface of the second layer 12 has a curvature with respect to the plane. The upper surface of the second layer 12 is, for example, the interface between the substrate 1 and the second layer 12. The lower surface of the second layer 12 is, for example, the interface between the second layer 12 and ambient air. The multiple second layers 12 overlap at least a portion of the light-emitting layer 41 in the first direction. A portion of the substrate 1 is positioned between the light-emitting layer 41 and the multiple second layers 12 in the first direction.

In the example illustrated in FIG. 8B, the second layer 12 is provided on the lower surface of the substrate 1; and the second layer 12 includes multiple protruding portions PP. The protruding portions PP protrude toward the fourth direction. The second layer 12 overlaps at least a portion of the light-emitting layer 41 in the first direction. A portion of the substrate 1 is positioned between the second layer 12 and the light-emitting layer 41 in the first direction.

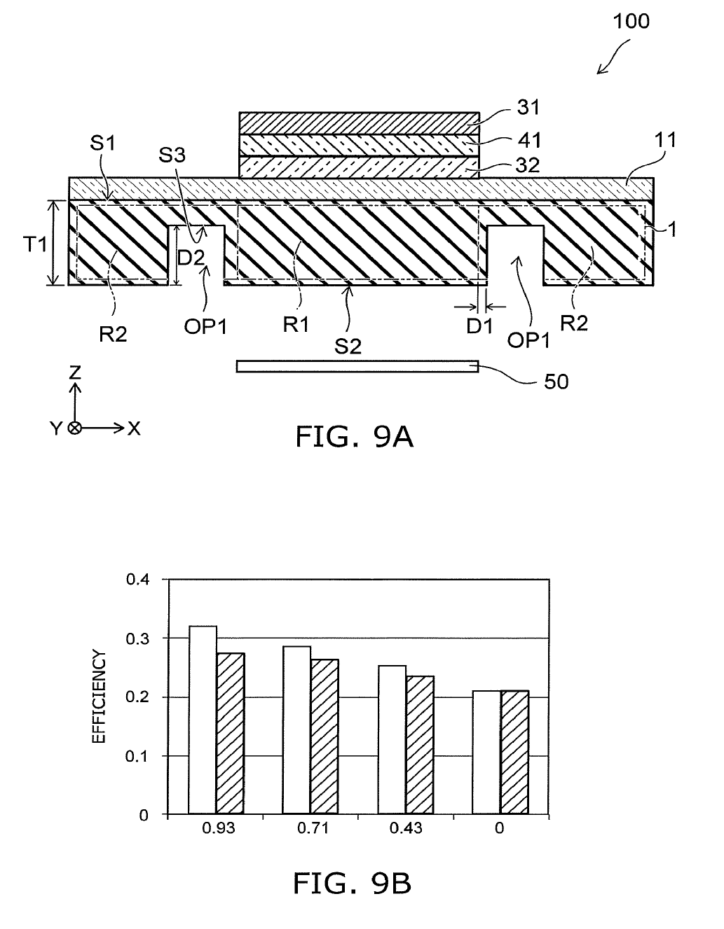

FIG. 9A and FIG. 9B illustrate simulation results of the light-emitting element according to the first embodiment. The simulation is performed using the following conditions.

The opening OP1 is multiply provided in the second region R2 as illustrated in FIG. 1; and each of the openings OP1 is aligned with the second direction or the third direction. A light detector 50 is provided at a position overlapping the light-emitting region 41a in the first direction. The first region R1 is positioned between the light detector 50 and the light-emitting layer 41. The surface area and configuration of the light detector 50 are the same as those of the light-emitting region 41a. The distance D1 in the second direction between the light-emitting region 41a and the opening OP1 is 0 mm or 0.1 mm.

In the simulation, the proportion of a depth D2 of the opening OP1 to the thickness T1 in the first direction of the substrate 1 is changed. In the light-emitting element illustrated in FIG. 9A, the depth D2 may be the distance in the first direction between the second surface S2 and the third surface S3.

In FIG. 9B, the horizontal axis is D2/T1; and the vertical axis is the efficiency. The efficiency is the proportion (L1/L0) of a light amount L0 to a light amount L1, where the light amount L0 is radiated from the light-emitting region 41a, and the light amount L1 passes through the second surface S2 and is emitted to the outside from the first region R1. The result in the case where the opening OP1 is not provided is D2/T1=0. The bars marked with diagonal lines illustrate the results in the case where D1=0.1 mm; and the bars not marked with diagonal lines illustrate the results in the case where D1=0 mm.

From the results illustrated in FIG. 9B, it can be seen that the efficiency is increased by providing the opening OP1. It can be seen that the efficiency increases as D2/T1 increases. It can be seen that the efficiency is higher for the case of D1=0 mm than for the case of D1=0.1 mm.

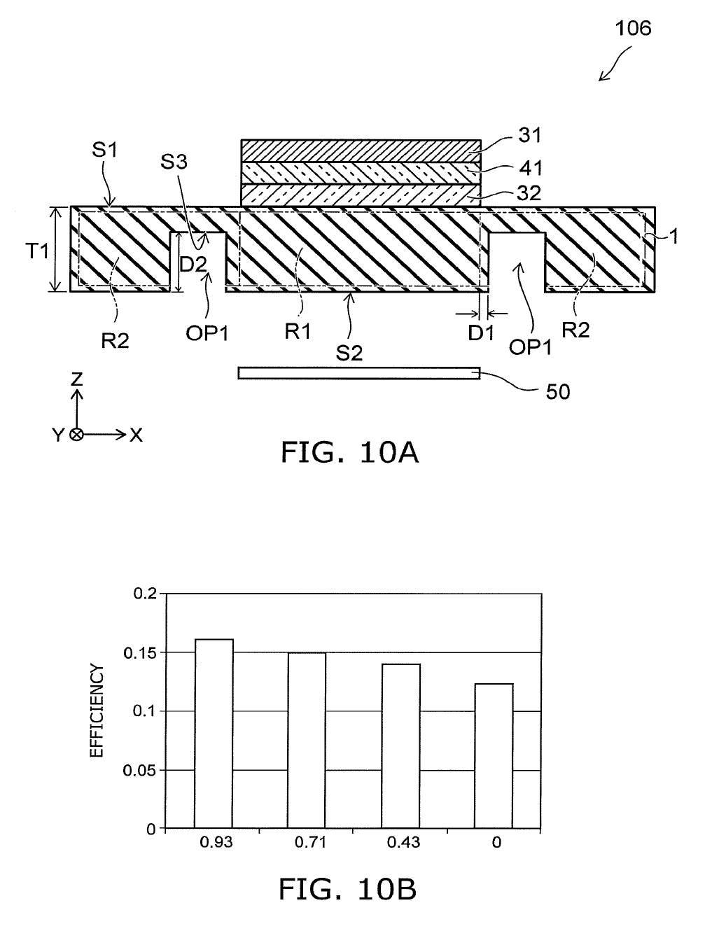

FIG. 10A and FIG. 10B illustrate other simulation results of the light-emitting element according to the first embodiment. Other than the light-emitting element 106 not including the first layer 11, the conditions of the simulation are similar to the conditions of the simulation illustrated in FIG. 9A and FIG. 9B. D1 is 0 mm.

From the results illustrated in FIG. 10B, it can be seen that the efficiency increases as D2/T1 increases. Comparing FIG. 9B and FIG. 10B, it can be seen that the efficiency is greatly increased in the case where the first layer 11 is provided.

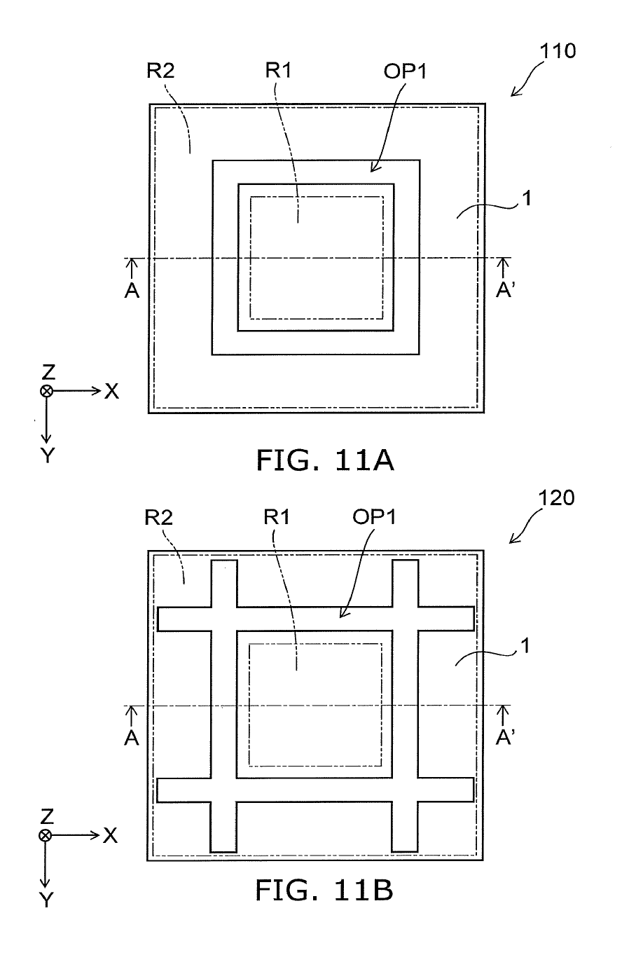

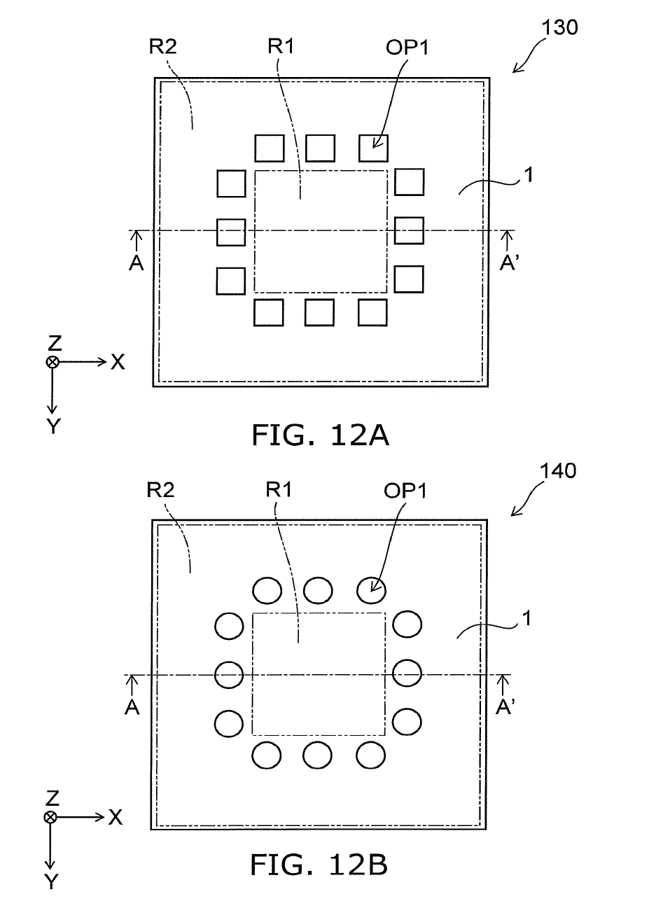

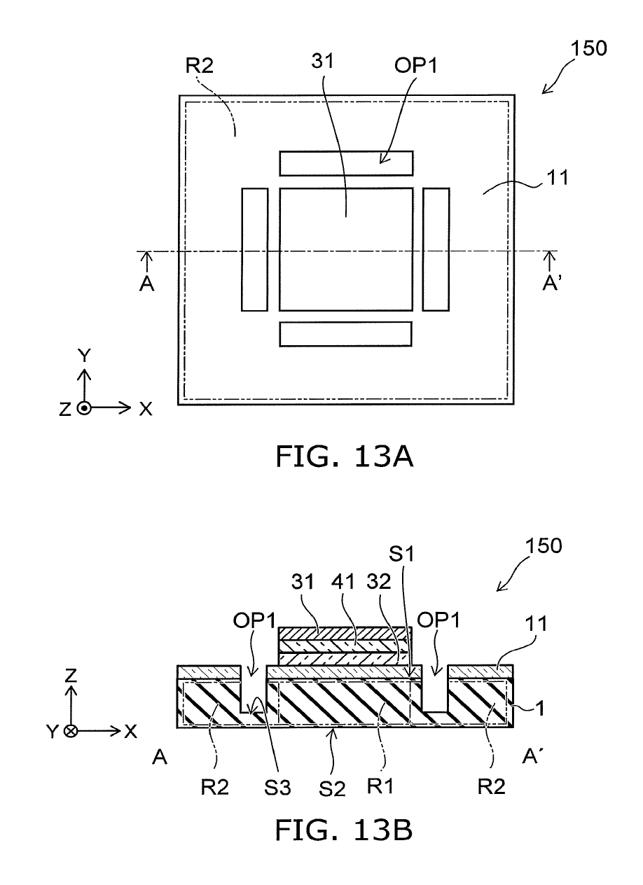

FIG. 11A, FIG. 11B, FIG. 12A, and FIG. 12B are schematic bottom views illustrating other examples of the light-emitting element according to the first embodiment. In a light-emitting element 110 illustrated in FIG. 11A, the opening OP1 surrounds the first region R1 along a plane perpendicular to the first direction. In a light-emitting element 120 illustrated in FIG. 11B, the opening OP1 surrounds the first region R1 along a plane perpendicular to the first direction and extends toward the outer edge of the substrate 1.

In a light-emitting element 130 illustrated in FIG. 12A, the multiple openings OP1 are provided around the first region R1 along a plane perpendicular to the first direction. In this example, the configurations of the openings OP1 in the plane perpendicular to the first direction are squares or rectangles.

Similarly, in a light-emitting element 140 illustrated in FIG. 12B, the multiple openings OP1 are provided around the first region R1. In this example, the configurations of the openings OP1 in the plane perpendicular to the first direction are circles or ellipses. In the examples illustrated in FIG. 12A and FIG. 12B, the openings OP1 are recesses.

The structures of the A-A′ cross sections in the light-emitting elements in the examples illustrated in FIG. 11A, FIG. 11B, FIG. 12A, and FIG. 12B are, for example, similar to the structure illustrated in FIG. 2.

FIG. 13A and FIG. 13B are schematic views illustrating another example of the light-emitting element according to the first embodiment. FIG. 13A is a schematic plan view; and FIG. 13B is a schematic cross-sectional view illustrating an A-A′ cross section of FIG. 13A.

In the light-emitting element 150, the opening OP1 is provided in the first surface S1 of the substrate 1. A portion of the substrate 1 is positioned between the second surface S2 and the opening OP1.

The structure of the opening OP1 illustrated in any of FIG. 11A, FIG. 11B, FIG. 12A, and FIG. 12B may be employed in the light-emitting element 150. The opening OP1 may be provided in the second region R2 to surround the first region R1. The multiple openings OP1 may be provided around the first region R1.

FIG. 14A and FIG. 14B are schematic views illustrating another example of the light-emitting element according to the first embodiment. FIG. 14A is a schematic plan view; and FIG. 14B is a schematic cross-sectional view illustrating an A-A′ cross section of FIG. 14A.

In the light-emitting element 160, the opening OP1 pierces through the substrate 1. The opening OP1 is a hole. In the light-emitting element 160 as well, similarly to the light-emitting element 100, the amount of the light radiated in the space overlapping the light-emitting region 41a in the first direction can be increased. The structure of the opening OP1 illustrated in FIG. 12A or FIG. 12B may be employed in the light-emitting element 160. The multiple openings OP1 may be provided around the first region R1.

### Second Embodiment

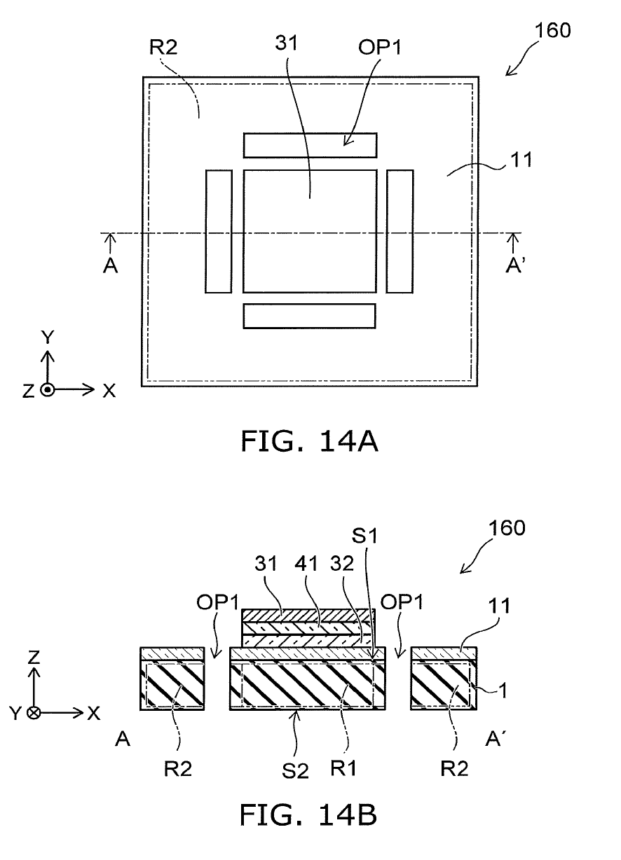

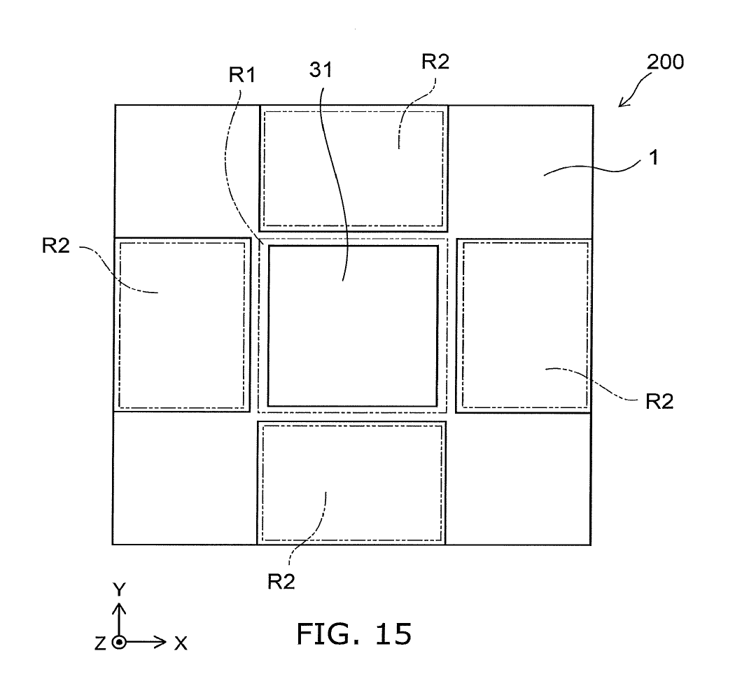

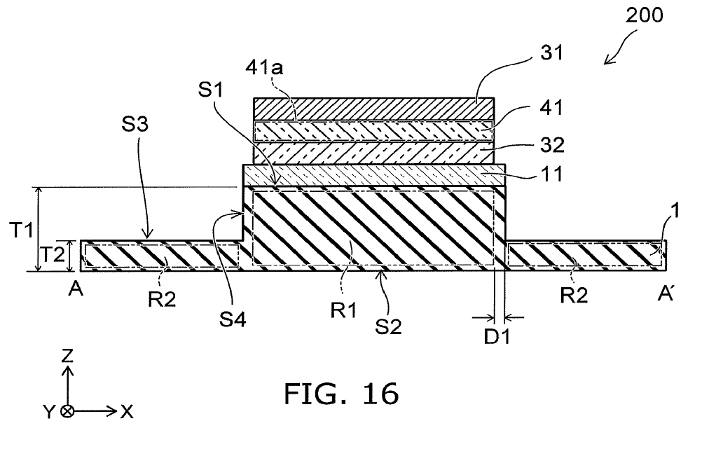

FIG. 15 is a schematic plan view illustrating an example of a light-emitting element according to a second embodiment. FIG. 16 is a schematic cross-sectional view illustrating an A-A′ cross section of FIG. 15.

As illustrated in FIG. 15 and FIG. 16, the light-emitting element 200 includes the substrate 1, the first layer 11, the second electrode 32, the light-emitting layer 41, and the first electrode 31. The substrate 1 includes the first region R1 and the second region R2. The first region R1 overlaps the light-emitting region 41a in the first direction. The second region R2 is provided in at least a portion around the first region R1 along a plane perpendicular to the first direction. For example, the multiple second regions R2 are provided around the first region R1.

The substrate 1 has the first surface S1, the second surface S2, the third surface S3, and a fourth surface S4. The first to third surfaces S1 to S3 are aligned with planes perpendicular to the first direction. The fourth surface S4 is aligned with the first direction. The position in the first direction of at least a portion of the fourth surface S4 is between the position in the first direction of the first surface S1 and the position in the first direction of the third surface S3.

A thickness T2 in the first direction of the second region R2 is thinner than the thickness T1 in the first direction of the first region R1. The thickness changes in a staircase configuration at the fourth surface S4 from the thickness T1 to the thickness T2 in the direction from the first region R1 toward the second region R2.

According to the embodiment, the light that is radiated from the light-emitting region 41a, is incident on the substrate 1, and travels toward the side surface of the substrate 1 can be reflected by the fourth surface S4 toward the first region R1. Therefore, the amount of the light radiated in the space overlapping the light-emitting region 41a in the first direction can be increased.

### Third embodiment

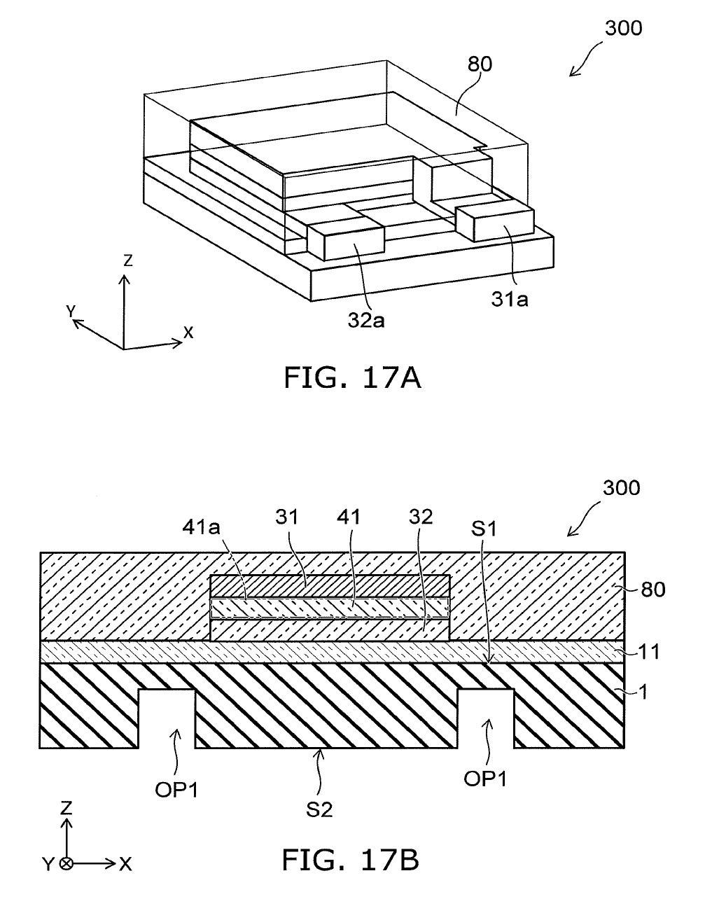

FIG. 17A and FIG. 17B are schematic views illustrating an example of a light-emitting element according to a third embodiment. FIG. 17A is a schematic perspective view; and FIG. 17B is a schematic cross-sectional view. The light-emitting element 300 includes, for example, the substrate 1, the first layer 11, the second electrode 32, the light-emitting layer 41, the first electrode 31, and a sealing portion 80. The first electrode 31 includes a first connection portion 31a. The second electrode 32 includes a second connection portion 32a.

Other than the connection portion of each electrode, the first electrode 31, the light-emitting layer 41, and the second electrode 32 are covered with the sealing portion 80. The light-emitting layer 41, a portion of the second electrode 32, and a portion of the first electrode 31 are provided between the substrate 1 and the sealing portion 80. The light-emitting layer 41, the portion of the second electrode 32, and the portion of the first electrode 31 are surrounded with the sealing portion 80 along a surface perpendicular to the first direction. The sealing portion 80 includes, for example, an insulating material such as silicon nitride, silicon oxynitride, etc. The sealing portion 80 may include a desiccant. For example, calcium oxide may be used as the desiccant. The sealing portion 80 can suppress reactions of the first electrode 31, the light-emitting layer 41, and the second electrode 32 with external moisture, etc.

In the light-emitting element 300, the various structures described in the first embodiment are employable as the structure of the opening OP1. By providing the opening OP1 in the second surface S2 as illustrated in FIG. 17B, it is possible to easily form the sealing portion 80 on the first surface S1.

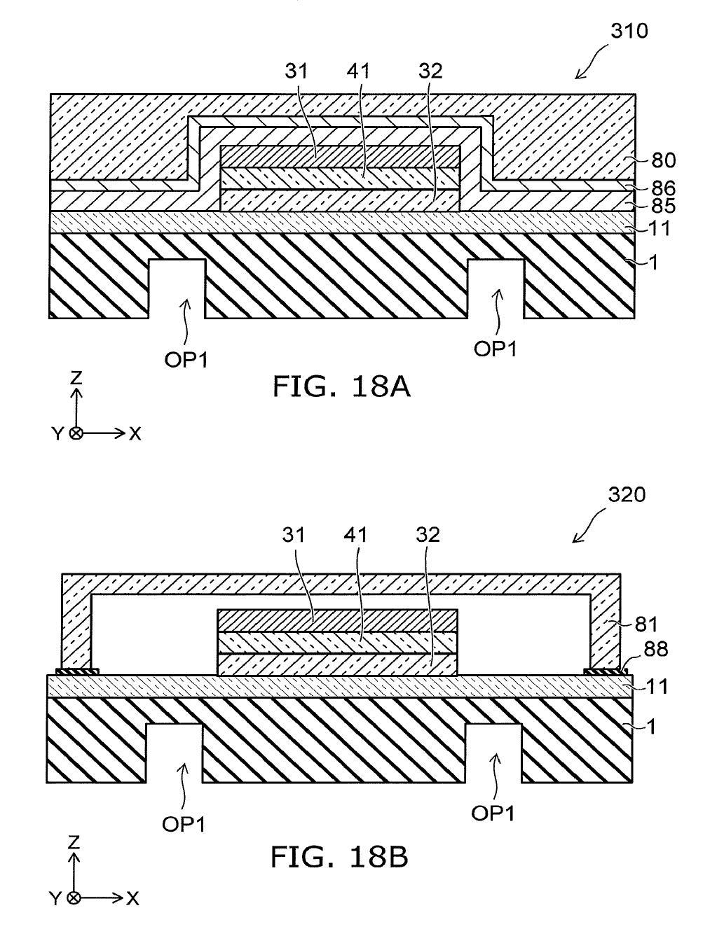

FIG. 18A and FIG. 18B are schematic cross-sectional views illustrating other examples of the light-emitting element according to the third embodiment. A light-emitting element 310 illustrated in FIG. 18A further includes a fifth layer 85 and a sixth layer 86 in addition to the components included in the light-emitting element 300.

For example, the sixth layer 86 includes a metal and reflects the light radiated from the light-emitting layer 41 toward the substrate 1. The fifth layer 85 includes, for example, an insulating material. The fifth layer 85 is provided between the sixth layer 86 and the first electrode 31 and between the sixth layer 86 and the second electrode 32. For example, the fifth layer 85 is provided to prevent the electrical contact between the first electrode 31 and the second electrode 32 due to the sixth layer 86.

By providing the sixth layer 86, it is possible to improve the proportion (L1/ L0) of the light amount L1 to the light amount L0, where the light amount L0 is radiated from the light-emitting region 41a, and the light amount L1 passes through the second surface S2 and is emitted to the outside from the first region R1.

Compared to the light-emitting element 300, for example, a light-emitting element 320 illustrated in FIG. 18B includes a sealing portion 81 instead of the sealing portion 80. The sealing portion 81 includes, for example, glass and is provided to be separated from the first electrode 31, the light-emitting layer 41, and the second electrode 32. For example, the sealing portion 81 is bonded to the first layer 11 by a bonding agent 88. The sealing portion 81 may be directly bonded to the substrate 1. For example, nitrogen gas is filled into the interior of the sealing portion 81.

### Fourth Embodiment

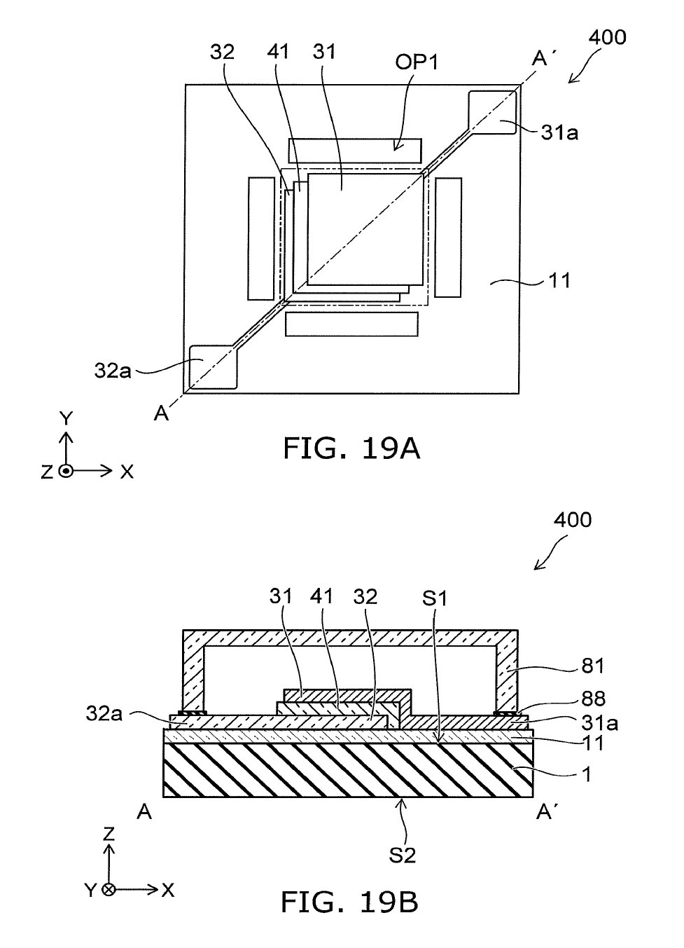

FIG. 19A and FIG. 19B are schematic views illustrating an example of a light-emitting element according to a fourth embodiment. FIG. 19A is a schematic plan view; and FIG. 19B is a schematic cross-sectional view illustrating an A-A′ cross section of FIG. 19A. The sealing portion 81 is not illustrated in FIG. 19A.

The light-emitting element 400 further includes, for example, the first connection portion 31a, the second connection portion 32a, and the sealing portion 81 in addition to the components included in the light-emitting element 150. For example, the sealing portion 81 is bonded to the first layer 11 by the bonding agent 88.

In the case where the opening OP1 is formed in the first surface S1, it is possible to easily perform the electrical connections of respectively between the connection portions and the electrodes by providing the multiple openings OP1 to be separated from each other as in the light-emitting element 400.

### Fifth Embodiment

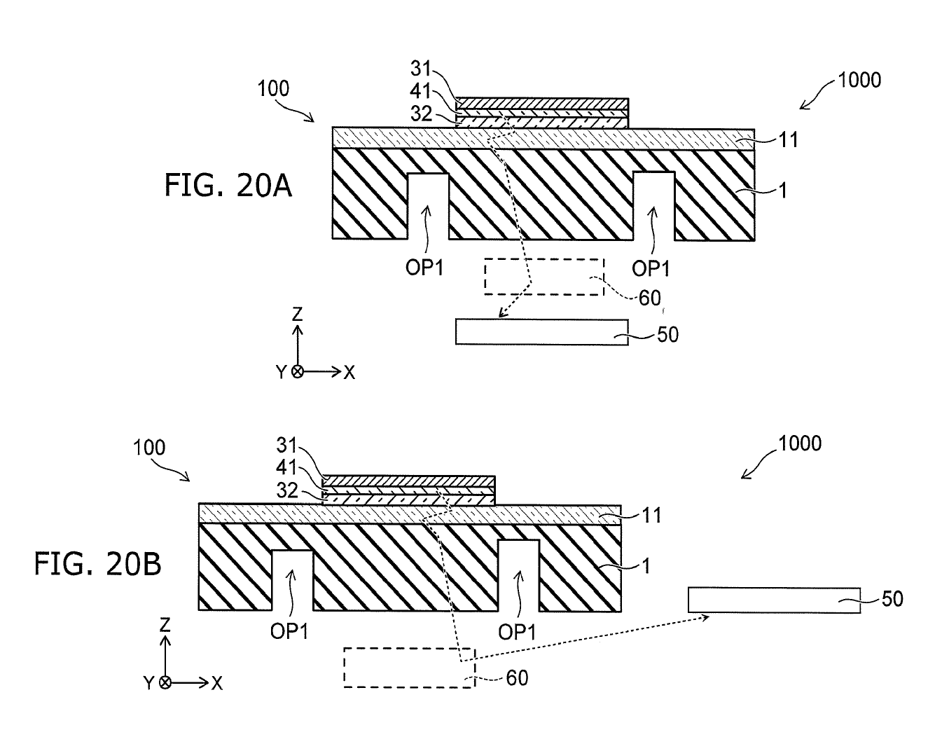

FIG. 20A and FIG. 20B are schematic cross-sectional views illustrating an example of a detection device according to a fifth embodiment. The detection device 1000 includes the light-emitting element 100, and the light detector 50 that detects the light radiated from the light-emitting layer 41. In FIG. 20A and FIG. 20B, examples of paths of the light radiated from the light-emitting layer 41 are illustrated by broken lines. The detection device 1000 may include another light-emitting element according to the embodiment instead of the light-emitting element 100.

As illustrated in FIG. 20A, for example, at least a portion of the light detector 50 overlaps at least a portion of the first electrode 31, at least a portion of the second electrode 32, and at least a portion of the light-emitting layer 41 in the first direction. For example, a detection object 60 is disposed between the light detector 50 and the light-emitting element 100.

Or, as illustrated in FIG. 20B, at least a portion of the light detector 50 may be arranged with at least a portion of the light-emitting element 100 in the second direction or the third direction. In such a case, the light is radiated from the light-emitting element 100, is incident on the detection object 60, and is reflected or scattered by the detection object 60. The light detector 50 detects the light that is reflected or scattered by the detection object 60.

Because the light-emitting element 100 is included in the detection device 1000, the amount of the light irradiated on the detection object 60 and incident on the light detector 50 can be increased; and it is possible to increase the detection sensitivity and the detection precision of the detection device 1000.

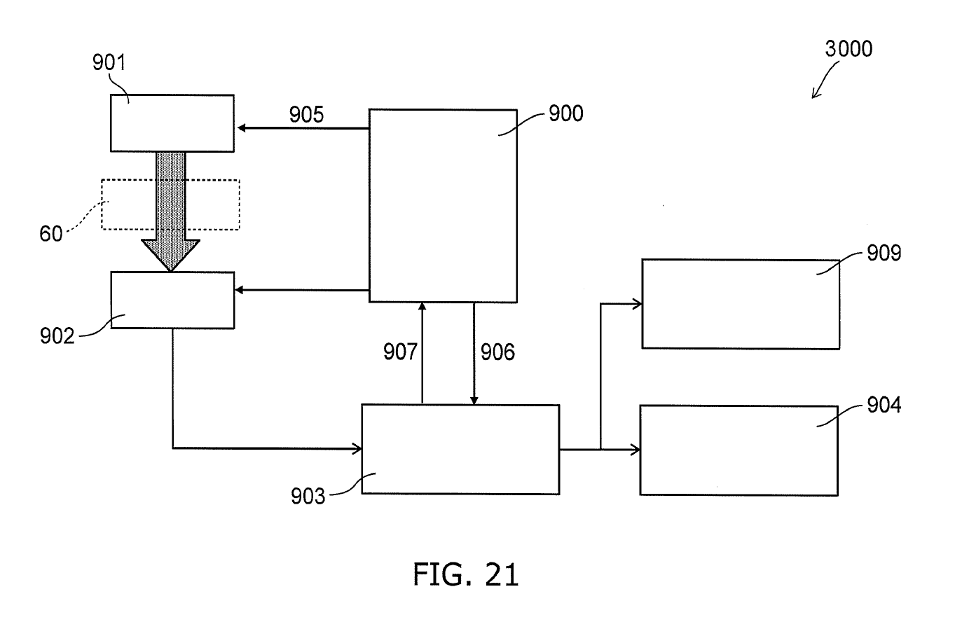

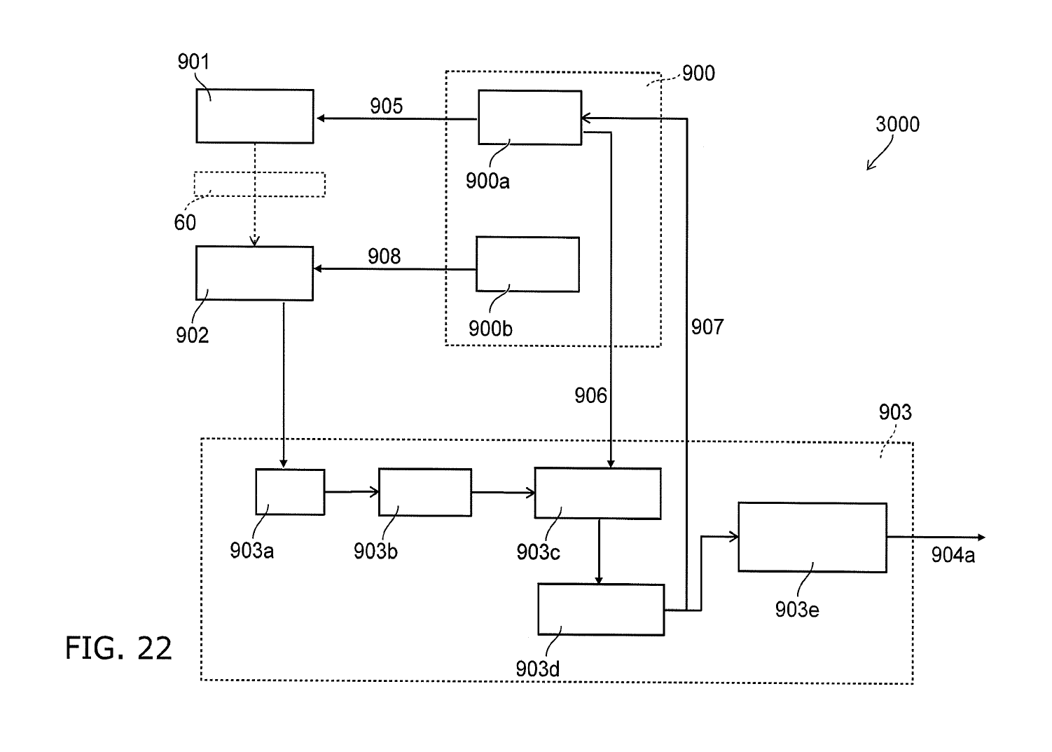

FIG. 21 and FIG. 22 are schematic views illustrating an example of a processing apparatus including the light-emitting element according to the embodiment. As illustrated in FIG. 21, the processing apparatus 3000 includes, for example, a controller 900, a light emitter 901, a light receiver 902, a signal processor 903, a recording device 904, and a display device 909.

The light emitter 901 includes any light-emitting element according to the embodiments. The light receiver 902 includes a light detector that detects the light emitted from the light emitter 901. The light emitter 901 that receives an input signal from the controller 900 emits light. The light that is emitted passes through the detection object 60, is reflected by the detection object 60, or is scattered by the detection object 60 and is detected by the light receiver 902. The light receiver 902 may receive a bias signal from the controller 900 to increase the detection sensitivity.

The signal that is detected by the light receiver 902 is output to the signal processor 903. The signal processor 903 receives the signal from the light receiver 902 and performs processing of the signal such as, for example, AC detection, signal amplification, noise removal, etc., as appropriate. To perform the appropriate signal processing, the signal processor 903 may receive a synchronization signal from the controller 900. A feedback signal for adjusting the light amount of the light emitter 901 may be transmitted to the controller 900 from the signal processor 903. The signal that is generated by the signal processor 903 is stored in the recording device 904; and the information is displayed by the display device 909.

The processing apparatus 3000 may not include the recording device 904 and the display device 909. In such a case, the signal that is generated by the signal processor 903 is output to, for example, a recording device and a display device outside the processing apparatus 3000.

The processing apparatus 3000 will now be described more specifically with reference to FIG. 22. As illustrated in FIG. 22, the light emitter 901 receives an input signal 905 including a DC bias signal or a pulse signal from the pulse generator 900a of the controller 900. The light that is emitted from the light emitter 901 passes through the detection object 60, or is reflected or scattered by the detection object 60, and is detected by the light receiver 902. The light receiver 902 may receive a bias signal from a bias circuit 900b of the controller 900. The signal that is detected by the light receiver 902 is input to the signal processor 903. After AC detection of the signal from the light receiver 902 is performed as necessary by the signal processor 903, the signal is amplified by an amplifier 903a; and unnecessary noise components are removed by a filter portion 903b. A signal synchronizer 903c receives the signal output from the filter portion 903b, and if appropriate, receives a synchronization signal 906 from the controller 900 and performs synchronization with the light.

The signal that is output from the signal synchronizer 903c is input to a signal shaper 903d. The processing apparatus 3000 may not include the signal synchronizer 903c. In such a case, the signal that is output from the filter portion 903b is input to the signal shaper 903d without going through the signal synchronizer 903c.

In the signal shaper 903d, the signal is shaped into the desired signal so that the appropriate signal processing is performed by a signal calculator 903e. For example, the signal shaping is performed by time averaging, etc. In the signal processor 903, the order of the AC detection and the processing performed by the processors is modifiable as appropriate. A calculated value 904a from the signal calculator 903e of the signal processor 903 is output to a recording device and a display device.

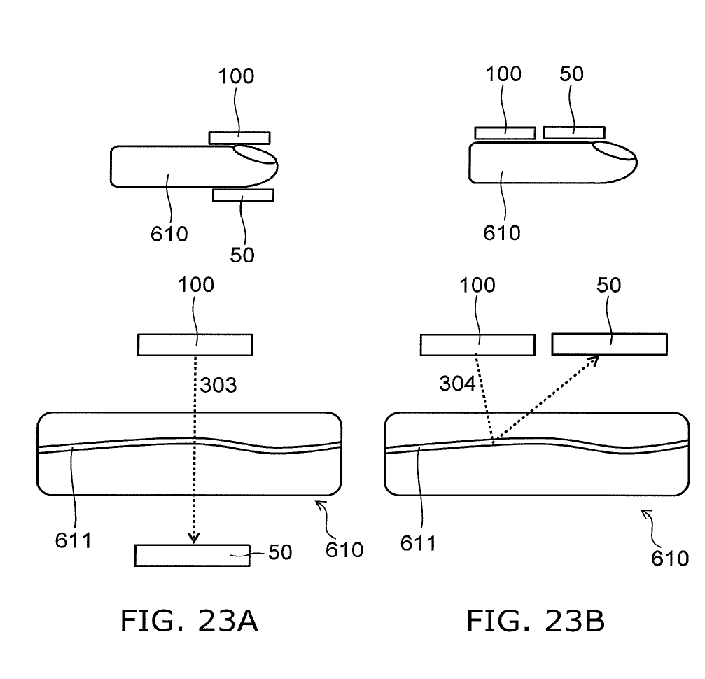

FIG. 23A to FIG. 26B are schematic views illustrating a pulse wave being measured using the light-emitting element according to the embodiment. The light-emitting element 100 is used in the example illustrated in FIG. 23A to FIG. 26B. Another light-emitting element according to any of the embodiments may be used instead of the light-emitting element 100.

FIG. 23A and FIG. 23B illustrate the detection of the pulse wave of a blood vessel 611 inside a finger 610. Other than the finger 610, the living body location may be selected arbitrarily to be an ear, a chest, an arm, etc.

In the example illustrated in FIG. 23A, light 303 that is emitted from the light-emitting element 100 passes through the blood vessel 611 and is detected by the light detector 50. In the example illustrated in FIG. 23B, light 304 that is emitted from the light-emitting element 100 is reflected or scattered by the blood vessel 611 and is detected by the light detector 50. At this time, the light detector 50 detects a signal reflecting the blood flow of the blood vessel 611. For example, the pulse is measured by the signal processor 903 shown in FIG. 21 and FIG. 22 performing signal processing of the signal that is detected.

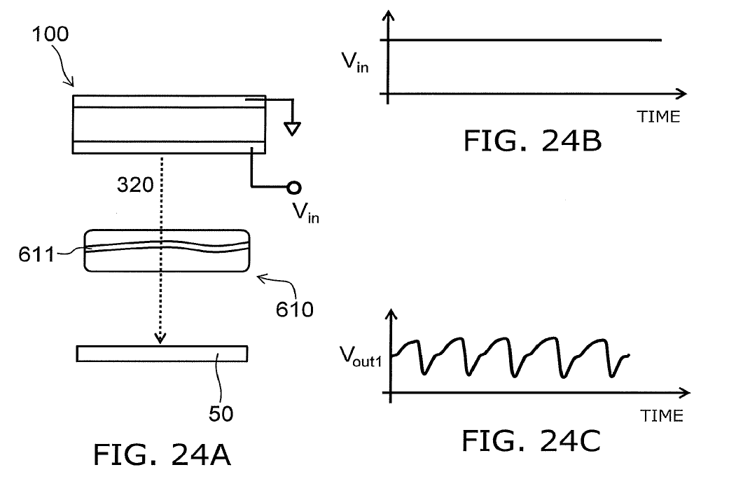

As illustrated in FIG. 24B, for example, a constant voltage is applied as an input signal Vᵢₙ to the first electrode 31 and the second electrode 32 of the light-emitting element 100. As illustrated in FIG. 24A, the light detector 50 detects the light passing through the finger 610 or the light reflected or scattered by the finger 610. At this time, as illustrated in FIG. 24C, the signal inside the blood is superimposed onto a signal Vₒᵤₜ detected by the light detector 50.

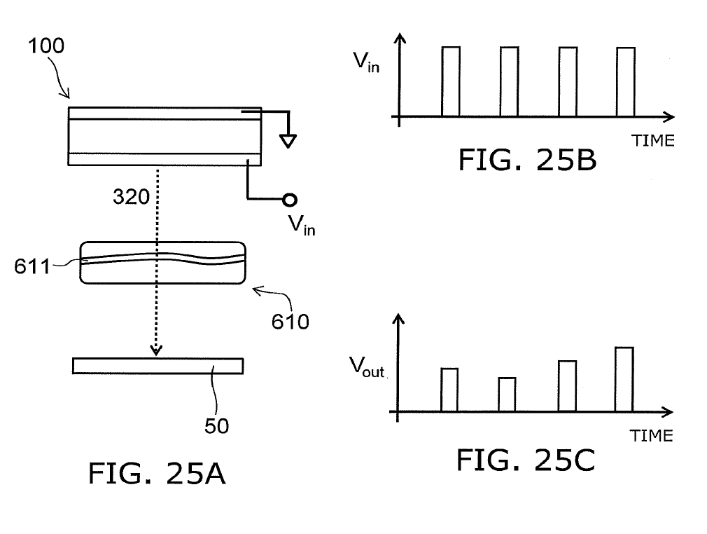

Or, as illustrated in FIG. 25A and FIG. 25B, the light may be radiated from the light-emitting element 100 by applying a pulse voltage as the input signal Vᵢₙ to the first electrode 31 and the second electrode 32 of the light-emitting element 100. As illustrated in FIG. 25C, the light on which the signal inside the blood is superimposed is detected by the light detector 50.

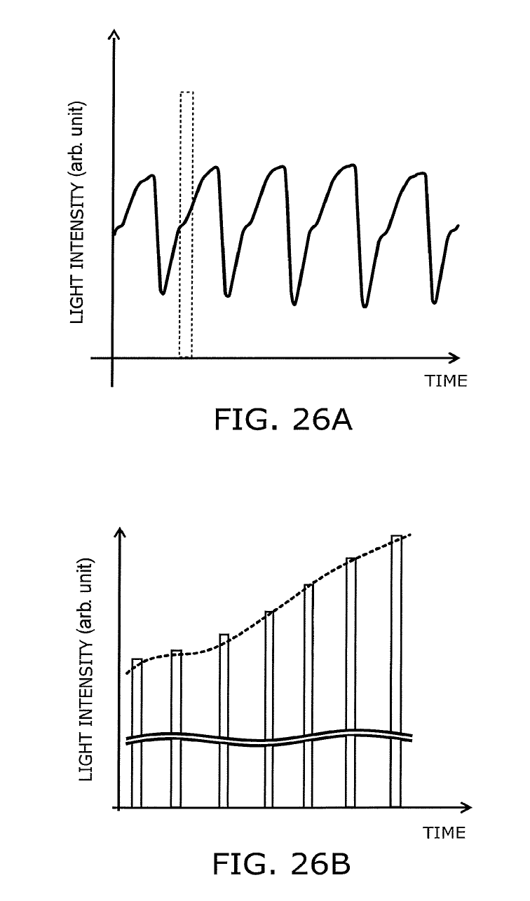

FIG. 26A and FIG. 26B illustrate an example of the optical signal detected in the case where a pulse voltage is applied as the input signal Vᵢₙ. FIG. 26B illustrates the enlarged portion surrounded with the broken line of FIG. 26A. In the case where the frequency of the pulse voltage applied to the light-emitting element 100 is sufficiently faster than the frequency of the pulse wave, the pulse wave signal is obtained by viewing only the optical signal of each light pulse as illustrated in FIG. 26A and FIG. 26B. Typically, the pulse wave is about 1 Hz; and the frequency of the pulse voltage may be set to, for example, 100 Hz to 100 KHz. Because the time that the light-emitting element 100 emits light is shorter for the configuration in which the pulse voltage illustrated in FIG. 25A to FIG. 26B is used than for the configuration in which the constant voltage illustrated in FIGS. 24A to 24C is used, this is advantageous in that the degradation of the light-emitting element 100 is suppressed; and the power consumption can be reduced.

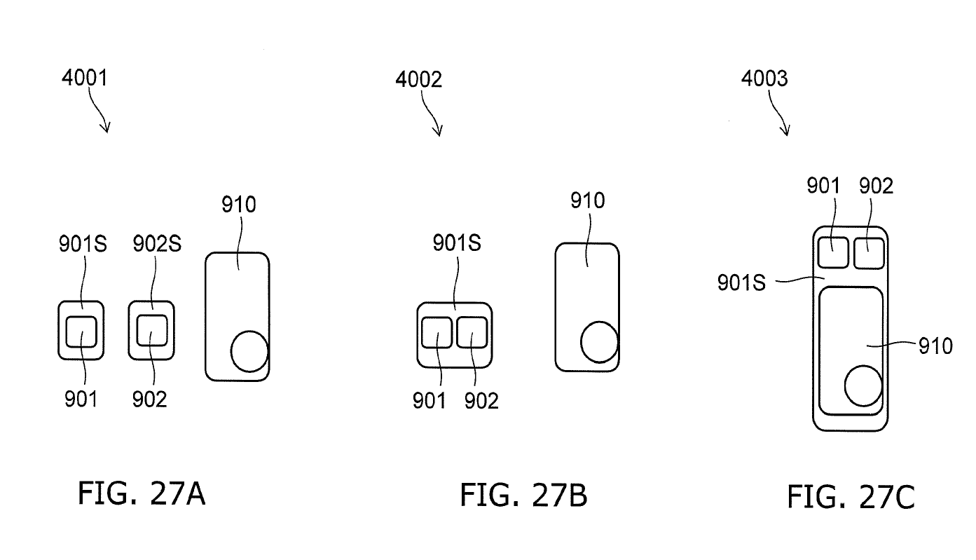

FIG. 27A to FIG. 27C are schematic views illustrating processing apparatuses including the light-emitting element according to the embodiment. The processing apparatuses 4001 to 4003 include the light emitter 901, the light receiver 902, and a controller/signal processor 910. The light emitter 901 includes the light-emitting element according to the embodiment.

In the processing apparatus 4001, the light emitter 901 is provided on a support substrate 901S; and the light receiver 902 is provided on a support substrate 902S. The processing apparatus 4001 has a configuration in which the light emitter 901, the light receiver 902, and the controller/signal processor 910 are provided independently from each other.

In the processing apparatus 4002, the light emitter 901 and the light receiver 902 are provided on a common support substrate 901S. In the processing apparatus 4003, the light emitter 901, the light receiver 902, and the controller/signal processor 910 are provided on a common support substrate 901S. The controller/signal processor 910 and one of the light emitter 901 or the light receiver 902 may be provided on a common support substrate.

Thus, various configurations are employable as the configuration of the processing apparatus.

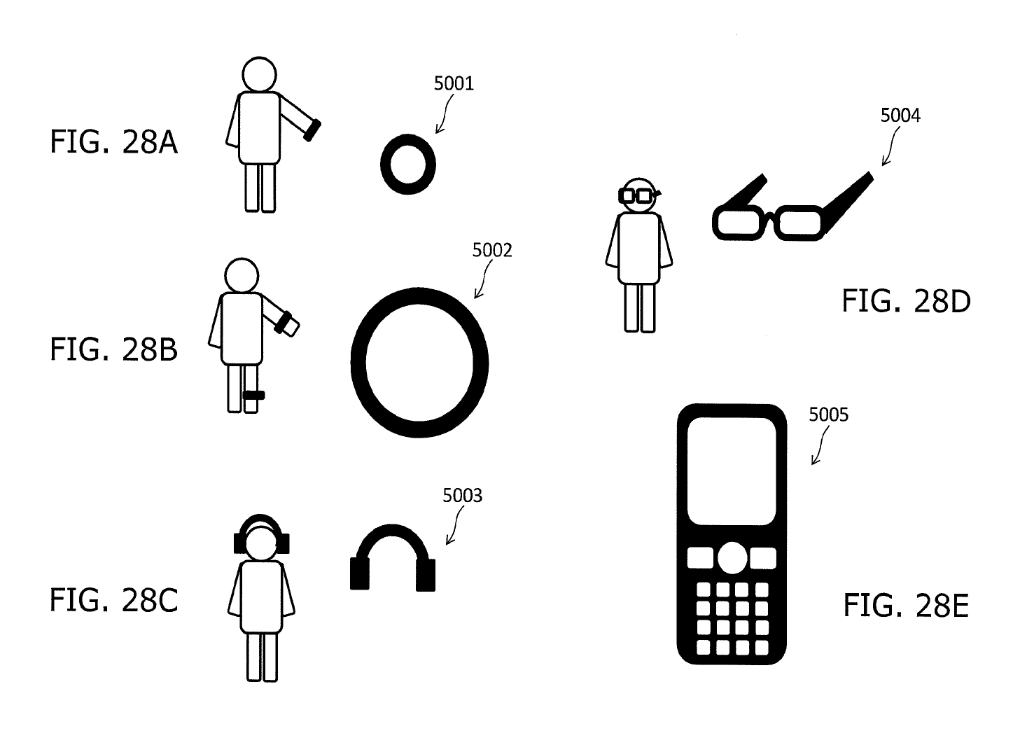

FIG. 28A to FIG. 28E are schematic views illustrating applications of processing apparatuses including the light-emitting element according to the embodiment. The processing apparatus in each example measures, for example, the pulse and/or the oxygen concentration of blood.

In the example illustrated in FIG. 28A, a processing apparatus 5001 is included in a finger ring. For example, the processing apparatus 5001 detects the pulse of a finger contacting the processing apparatus 5001. In the example illustrated in FIG. 28B, a processing apparatus 5002 is included in an arm band. For example, the processing apparatus 5002 detects the pulse of an arm or a leg contacting the processing apparatus 5002.

In the example illustrated in FIG. 28C, a processing apparatus 5003 is included in an earphone. In the example illustrated in FIG. 28D, a processing apparatus 5004 is included in eyeglasses. For example, the processing apparatuses 5003 and 5004 detect the pulse of an ear lobe. In the example illustrated in FIG. 28E, a processing apparatus 5005 is included in a button, a screen, etc., of a mobile telephone or a smartphone. For example, the processing apparatus 5005 detects the pulse of a finger touching the processing apparatus 5005.

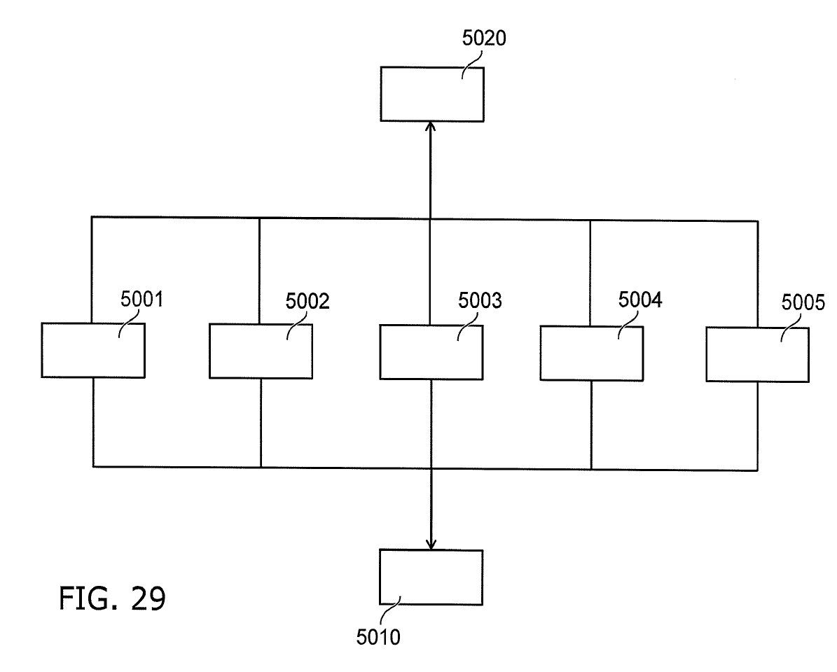

FIG. 29 is a schematic view illustrating a system including the processing apparatuses illustrated in FIGS. 28A to 28E.

For example, the processing apparatuses 5001 to 5005 transmit the measured data to a device 5010 such as a desktop PC, a notebook PC, a tablet terminal, etc., by a wired or wireless method. Or, the processing apparatuses 5001 to 5005 may transmit the data to a network 5020.

The data that is measured by the processing apparatuses can be monitored by utilizing the device 5010 or the network 5020. Or, monitoring or statistical processing may be performed by analyzing the measured data by using an analysis program, etc.

In the case where the measured data is a pulse or an oxygen concentration of blood, the summary of the data may be performed at any time interval. For example, the data that is summarized is utilized for health care. At a hospital, for example, the data is utilized for continuous monitoring of the health condition of a patient.

According to the embodiments recited above, a light-emitting element, a detection device, and a processing apparatus suited to the detection of a faint signal such as a pulse wave or the like can be provided.

In the specification of the application, “perpendicular” and “parallel” refer to not only strictly perpendicular and strictly parallel but also include, for example, the fluctuation due to manufacturing processes, etc. It is sufficient to be substantially perpendicular and substantially parallel.

Hereinabove, embodiments of the invention are described with reference to specific examples. However, the invention is not limited to these specific examples. For example, one skilled in the art may similarly practice the invention by appropriately selecting specific configurations of components included in the light-emitting element, the detection device, and the processing apparatus such as the substrate 1, the first layer 11, the second layer 12, the first electrode 31, the second electrode 32, the light-emitting layer 41, the third layer 43, the fourth layer 44, the light detector 50, the sealing portion 80 and 81, the fifth layer 85, the sixth layer 86, the support portion 121, the particles 122, the controller 900, the light receiver 902, the signal processor 903, the recording device 904, and the display device 909, etc., from known art; and such practice is within the scope of the invention to the extent that similar effects can be obtained.

Further, any two or more components of the specific examples may be combined within the extent of technical feasibility and are included in the scope of the invention to the extent that the purport of the invention is included.

Moreover, all light-emitting elements, all detection devices, and all processing apparatuses practicable by an appropriate design modification by one skilled in the art based on the light-emitting elements, the detection devices, and the processing apparatuses described above as embodiments of the invention also are within the scope of the invention to the extent that the spirit of the invention is included.

Various other variations and modifications can be conceived by those skilled in the art within the spirit of the invention, and it is understood that such variations and modifications are also encompassed within the scope of the invention.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the invention.

What is claimed is:

1. A light-emitting element, comprising:
a substrate, the substrate being light-transmissive;
a first electrode;
a second electrode provided between the first electrode and a portion of the substrate, the second electrode being light-transmissive; and
a light-emitting layer provided between the first electrode and the second electrode,
the substrate including
a first region overlapping at least a portion of the light-emitting layer in a first direction, the first direction being from the second electrode toward the first electrode, and
a second region provided around the first region along a plane perpendicular to the first direction,
the substrate having an opening provided in at least a portion of the second region.

2. The element according to claim 1, wherein at least a portion of the opening is aligned with a boundary between the first region and the second region.

3. The element according to claim 1, wherein
the opening includes a first end and a second end,
the light-emitting layer includes a third end and a fourth end,
a direction from the first end toward the second end is aligned with a second direction crossing the first direction,
a direction from the third end toward the fourth end is aligned with the second direction,
a position in the second direction of the first end and a position in the second direction of the fourth end are between a position in the second direction of the third end and a position in the second direction of the second end, and
a direction from the first end toward the fourth end is aligned with the first direction.

4. The element according to claim 1, wherein the opening surrounds the first region along the plane perpendicular to the first direction.

5. The element according to claim 1, wherein
the substrate has a plurality of the openings, and
the plurality of openings is provided around the first region along the plane perpendicular to the first direction.

6. The element according to claim 1, wherein
the substrate has a first surface on the first electrode side, and
a portion of the substrate is provided between the opening and the first surface.

7. The element according to claim 1, wherein
the substrate has a first surface and a second surface, the first surface being on the first electrode side, the second surface being on a side opposite to the first surface, and
a portion of the substrate is provided between the opening and the second surface.

8. The element according to claim 1, wherein the opening pierces through the substrate along the first direction.

9. The element according to claim 1, further comprising a first layer, the first layer being light-transmissive,
at least a portion of the first layer being provided between the first electrode and at least a portion of the substrate,
the first layer being configured to modify a travel direction of light incident on the first layer.

10. The element according to claim 9, wherein the first layer scatters the light incident on the first layer.

11. The element according to claim 9, wherein
the first layer includes a plurality of particles, and
a diameter of at least one of the plurality of particles is greater than 1/10 of a peak wavelength of light radiated from the light-emitting layer.

12. The element according to claim 9, wherein
the first layer includes a first portion and a second portion,
the first portion is provided around the second portion along the plane perpendicular to the first direction, and
a refractive index of the second portion is lower than a refractive index of the first portion.

13. The element according to claim 1, wherein the light-emitting layer includes at least one of an organic substance or an organic compound.

14. The element according to claim 1, wherein
the light-emitting layer includes a light-emitting region positioned between the first electrode and the second electrode in the first direction, and
a thickness T in the first direction of the substrate, a distance D in a second direction perpendicular to the first direction between the light-emitting region and the opening, and a refractive index n of the substrate satisfy
D<T/tanθ, and
θ=arctan(1/n).

15. The element according to claim 14, wherein D≦T/2tanθ is satisfied.

16. The element according to claim 1, further comprising a sealing portion,
the light-emitting layer being provided between a portion of the sealing portion and a portion of the substrate in the first direction,
the light-emitting layer being surrounded with the sealing portion along the plane perpendicular to the first direction.

17. A detection device, comprising:
the light-emitting element according to claim 1; and
a light detector detecting light radiated from the light-emitting element.

18. The device according to claim 17, wherein at least a portion of the light detector overlaps at least a portion of the light-emitting element in the first direction.

19. The device according to claim 17, wherein at least a portion of the light detector overlaps at least a portion of the light-emitting element in a second direction perpendicular to the first direction.

20. A processing apparatus, comprising:
the detection device according to claim 16; and
a processor receiving and processing a signal detected by the detection device.
